(19) United States
(12) Patent Application Publication
(10) Pub. No.: US 2009/0149314 A1
(43) Pub. Date: Jun. 11, 2009

(54) MIXED OXIDE CATALYSTS

(75) Inventors: Martin Ernst, Heidelberg (DE); Thilo Hahn, Kirchheimbolanden (DE); Johann-Peter Melder, Boehl-Iggelheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/282,195

(22) PCT Filed: Mar. 2, 2007
(86) PCT No.: PCT/EP07/52013

§ 371 Date: Sep. 9, 2008

(30) Foreign Application Priority Data
Mar. 10, 2006 (EP) 06110972.4

(51) Int. Cl. B01J 38/48; B01J 23/78; C07C 211/04; C07C 211/03; C07C 35/08; C07C 13/26; C07C 211/09

(52) U.S. Cl. 502/22; 502/330; 502/328; 564/511; 564/463; 568/835; 585/20; 564/509

(57) ABSTRACT

Catalysts which are prepared by reducing catalyst precursors which comprise a) cobalt and b) one or more elements of the alkali metal group, of the alkaline earth metal group, of the group consisting of the rare earths or zinc or mixtures thereof, the elements a) and b) being present at least partly in the form of their mixed oxides, and a process for the preparation of these catalysts and the use thereof for the hydrogenation of unsaturated organic compounds. Furthermore, a process for regenerating these catalysts by treatment of the catalyst with a liquid is described.

The present invention relates to catalysts which are prepared by reducing catalyst precursors which comprise a) cobalt and b) one or more elements of the alkali metal group, of the alkaline earth metal group, of the group consisting of the rare earths or zinc or mixtures thereof, the elements a) and b) being present at least partly in the form of their mixed oxides. The present invention furthermore relates to processes for the preparation of these catalysts and the use there of for hydrogenation. The present invention also relates to a process for regenerating these catalysts.

Further embodiments of the invention are described in the claims, the description and the examples. Of course, the abovementioned features of the subject matter according to the invention and those still to be explained below can be used not only in the respective stated combination but also in other combinations without departing from the scope of the invention.

Cobalt catalysts are as a rule prepared by calcination and reduction of catalyst precursors, such as cobalt hydroxide, cobalt nitrate and cobalt oxide or are used in the form of cobalt sponge catalysts (Raney cobalt) in hydrogenation reactions.

The hydrogenation of organic nitriles with Raney catalysts is frequently carried out in the presence of basic alkali metal or alkaline earth metal compounds, as described in U.S. Pat. No. 3,821,305, U.S. Pat. No. 5,874,625, U.S. Pat. No. 5,151,543, U.S. Pat. No. 4,375,003, EP-A-0316761, EP-A-0913388 and U.S. Pat. No. 6,660,887.

Cobalt-containing catalysts can furthermore be prepared by reducing cobalt-oxide, cobalt hydroxide or cobalt carbonate. DE-A-3403377 describes catalysts which comprise metallic cobalt particles and/or nickel particles which are obtainable from cobalt oxide particles and/or nickel oxide particles by contact with hydrogen. According to this disclosure, the content of alkali and/or alkaline earth metal is advantageously less than 0.1% by weight. EP-B-0742045 describes cobalt catalysts which are prepared by calcination of the oxides of the elements cobalt (55-98% by weight), phosphorus (from 0.2 to 15% by weight), manganese (from 0.2 to 15% by weight) and alkali metal (from 0.05 to 5% by weight) and subsequent reduction in a hydrogen stream. Cobalt catalysts which are obtainable by precipitation of cobalt carbonate from an aqueous solution of a cobalt salt and subsequent reduction with hydrogen are described in EP-A-0 322 760. In addition, these catalysts may comprise from 0.25 to 15% by weight, based on the total mass of the catalyst, of SiO₂, MnO₂, ZrO₂, Al₂O₃ and MgO in the form of the oxides, hydroxides or hydrated oxides. Hydrogenation catalysts which consist of one or more oxides of the elements Fe, Ni, Mn, Cr, Mo, W and P and one or more oxides of the alkali metal, alkaline earth metal and rare earth group are described in EP-B-0 445 589. According to the disclosure, the oxides are present partly as metals after reduction.

By means of this invention, it was intended to provide improved catalysts for hydrogenation which permit advantages over conventional processes. Thus, as small amounts as possible of metals, such as, for example, aluminum in the case of skeletal catalysts or alkaline promoters, such as lithium, should dissolve out of the catalyst, since this leads to declining stability and deactivation of the catalyst. Aluminates which form under basic conditions from the aluminum which has dissolved out can, in the form of solid residues, lead to blockages and deposits and cause the decomposition of a desired product. A further aim of the present invention was to provide catalysts which permit the hydrogenation of organic compounds under simplified reaction conditions. Thus, it was intended to provide catalysts which make it possible to carry out the hydrogenation reaction at lower pressures. Furthermore, it was intended to provide hydrogenation processes which can be carried out in the absence of water, ammonia and aqueous base.

The aim of this invention was furthermore to provide a hydrogenation process which permits the hydrogenation of nitrites to primary amines with high selectivity. Accordingly, the catalysts described at the outset were found.

According to the invention, the catalyst is obtainable by reducing a catalyst precursor containing a) cobalt and b) one or more elements of the alkali metal group, of the alkaline earth metal group, of the group consisting of the rare earths or zinc or mixtures thereof, the elements a) and b) being present at least partly in the form of their mixed oxides.

In a mixed oxide, in addition to cobalt and oxygen, the crystal lattice also comprises at least one further element b) from the group consisting of alkali or alkaline earth metals or the group consisting of the rare earths or zinc. Thus, b) may be lithium, sodium, potassium, rubidium, cesium, beryllium, magnesium, calcium, strontium, barium, radium, scandium, yttrium, lanthanum, cerium, praseodymium, neodymium, promethium, samarium, europium, gadolinium, terbium, dysprosium, holmium, erbium, thulium, ytterbium or zinc, preferably lithium, sodium, potassium, magnesium, calcium or zinc or a mixture of two or more of said elements.

Depending on the ratios of cobalt to the element b),
1. the element b) can occupy a lattice site (substitution solid solution) or an interstitial site (interstitial solid solution) instead of cobalt,
2. cobalt can occupy a lattice site or an interstitial site instead of the element b) or
3. cobalt and the element b) can form with oxygen a common crystal lattice which resembles none of the parent compounds.

In this description, the designation mixed oxide also expressly includes so-called “solid solutions”, i.e. continuous series of solid solutions. A mixture of oxides or an oxide mixture differs from the mixed oxide present according to the invention in that the crystal structures of cobalt oxide and of the oxides of the elements b) are present side by side in more or less fine distribution in a mixture of oxides or an oxide mixture. That the mixed oxide according to the invention is present can be detected analytically, for example by means of X-ray diffractometry. Comparative or reference spectra are to be found in crystallographic databases [ICSD (Inorganic Crystal Structure Database), Bergerhoff et al, University of Bonn (Germany) or Powder Diffraction File, Berry et al., International Centre for Diffraction Data (ICDD), Swarthmore (USA)].

The catalyst precursors which are used for the preparation of the catalysts according to the invention are present, as explained above, partly as mixed oxide, comprising cobalt and at least one of the abovementioned elements b). Preferably, the catalyst precursors are present partly as mixed oxides of Co and Li, as mixed oxides of Co and Na, as mixed oxides of Co and K, as mixed oxides of Co and Rb, as mixed oxides of Co and Cs, as mixed oxides of Co and Be, as mixed oxides of Co and Mg, as mixed oxides of Co and Ca, as mixed oxides of Co and Sr, as mixed oxides of Co and Ba, as mixed oxides of Co and La, as mixed oxides of Co and Y and as mixed oxides of Co and Zn. Particularly preferably, the catalyst precursors are present partly as mixed oxides of Co and Li, as mixed oxides of Co and Mg and as mixed oxides of Co and Zn, and very particularly preferably the catalyst precursors are present partly as mixed oxides of Co and Li and as mixed oxides of Co and Mg.

In a further preferred embodiment, the catalyst precursors which are used for the preparation of the catalysts according to the invention are present partly as mixed oxides of Li, Na and Co, as mixed oxides of Li, K and Co, as mixed oxides of Li, Mg and Co, as mixed oxides of Li, Ca and Co, as mixed oxides of Na, Mg and Co, as mixed oxides of K, Mg and Co, as mixed oxides of Na, Ca and Co and as mixed oxides of K, Ca and Co.

In a preferred embodiment, it is possible to reduce catalyst precursors which comprise one or more compounds of the empirical formula M′ₓM″_{y}CO_{z}O_{(x/2+y+z*1.5)}, where x=0 or x=0.1 to 1, y=0 or y=0.1 to 1 and z=0.1 to 1, and x and y cannot simultaneously be zero, and M′ is at least one element of the alkali metal group and M″ is at least one element of the alkaline earth metal group or zinc.

The catalyst precursor having the empirical formula LiCoO₂ (lithium cobaltite) is particularly preferred. LiCoO₂ may be present in the form of the low-temperature phase (LT-LiCoO₂), the high-temperature phase (HT-LiCoO₂) or a mixture of the two. In a further preferred embodiment, lithium cobaltite which is obtained by the recycling of batteries is used as the catalyst precursor. Furthermore, continuous solid solution series of Co oxide and Mg oxide having the formula MgₐCo_{b}O₁ are suitable as catalyst precursors, where 0<a<1 and 0<b<1 and a+b=1.

According to the invention, the catalyst precursors are present partly in the form of their mixed oxides. The catalyst precursors can, however, also be present exclusively in the form of their mixed oxides. Preferably, the proportion of cobalt in the catalyst precursor which is present in the form of mixed oxides is at least 10 mol %, advantageously at least 20 mol % and particularly preferably at least 30 mol %, based in each case on the cobalt present altogether in the catalyst precursor. It is also possible for the catalyst precursor to comprise one or more additional components in addition to one or more mixed oxides. Oxides of elements may be present as additional components. Oxides of the elements of the first to fifth main group or oxides of the elements of the third to eighth subgroup may be suitable as oxides of the elements, in particular oxides of the elements Co, Ni, Cu, Mn, P, Cr, Ag, Fe, Zr, Al, Ti, Li, Na, K, Mg, Ca, Zr, La or Y.

The catalyst precursor may comprise one or more doping element. Suitable doping elements are the elements of the 3rd to 8th subgroup of the Periodic Table of the Elements (in the version of 10.03.2005 of IUPAC (http://www.iupac.org/reports/periodic_table/IUPAC_Periodic_Table-3Oct05.pdf)), and the elements of the third, fourth and fifth main group. Preferred doping elements are Fe, Ni, Cr, Mn, P, Ti, Nb, V, Cu, Ag, Pd, Pt, Rh, Ir, Ru and Au. The doping elements are preferably present in amounts of not more than 10% by weight, for example from 0.1 to 10% by weight, particularly preferably in amounts of from 1 to 5% by weight, based in each case on the catalyst precursor used.

Catalyst precursors can be prepared in general by thermal treatment of the corresponding compounds of cobalt and one or more compounds of the alkali metal group, of compounds of the alkaline earth metal group, of compounds from the group consisting of the rare earths or of compounds of zinc, for example the nitrates, carbonates, hydroxides, oxides, acetates, oxalates or citrates. Thermal treatment may be understood, for example, as the fusing together or calcination of the abovementioned compounds. The thermal treatment of the abovementioned compounds, such as the nitrates, carbonates, hydroxides or oxides, can be effected in the air. In a preferred embodiment, the thermal treatment, in particular of the carbonates, is effected under an inert gas atmosphere. Suitable inert gas is, for example, nitrogen, carbon dioxide, helium, neon, argon, xenon, krypton or a mixture of said inert gases. Nitrogen is preferably suitable. The preparation of the catalyst precursors by thermal treatment of the abovementioned compounds under an inert gas atmosphere has the advantage that the subsequent reduction of the catalyst precursor can directly follow the thermal treatment described above. If the catalyst precursor is not prepared under an inert gas atmosphere, an additional blanketing step should be effected before the reduction. In the blanketing step, troublesome compounds, such as atmospheric oxygen, which may react with the reducing agent in the reduction, can be removed, for example by gassing the catalyst precursor with inert gas or by repeated evacuation and aeration with inert gas.

A further process for the preparation of the catalyst precursors is precipitation from water-soluble cobalt compounds and at least one or more elements from the group consisting of the water-soluble alkali metal compounds, of the water-soluble alkaline earth metal compounds, of the water-soluble compounds of the rare earths and of the water-soluble zinc compounds by addition of an alkaline solution and subsequent drying and calcination.

Processes for the preparation of LiCoO₂ are described, for example, in Antolini [E. Antolini, Solid State Ionics, 159-171 (2004)] and Fenton et al. [W. M. Fenton, P. A. Huppert, Sheet Metal Industries, 25 (1948), 2255-2259).

Thus, LiCoO₂ can be prepared by thermal treatment of the corresponding lithium and cobalt compounds, such as the nitrates, carbonates, hydroxides, oxides, acetates or oxalates.

Furthermore, LiCoO₂ can be obtained by precipitation from water-soluble lithium and cobalt salts by addition of an alkaline solution and subsequent calcination.

LiCoO₂ can also be obtained by the sol-gel process.

LiCoO₂ can, as described by Song et al. [S. W. Song, K. S. Han, M. Yoshimura, Y. Sata, A. Tatsuhiro, Mat. Res. Soc. Symp. Proc, 606, 205-210 (2000)], also be obtained by a hydrothermal treatment of cobalt metal with aqueous LiOH solutions.

According to the invention, LiCoO₂ which is obtained by the recycling of batteries can also be used as a catalyst precursor. A method for the recycling or recovery of lithium cobaltite from old batteries can be derived, for example, from CN 1594109. By mechanically opening the battery and dissolving away aluminum constituents with concentrated NaOH, an LiCoO₂-rich filter cake can be obtained.

After the synthesis of the oxidic catalyst precursor, a wash step or a wash step with subsequent drying can follow prior to the reduction. Impurities, byproducts or unconverted starting materials can be removed by the wash step.

The catalyst precursor may, as described above, comprise one or more doping elements.

These dopants can be introduced by adding metal complexes and metal salts, such as metal carbonates and metal oxides, or the metals themselves during the preparation of the catalyst precursor by fusing together the corresponding oxides or carbonates or mixtures thereof. It is also possible for the dopants to be introduced in the preparation via a precipitation reaction as water-soluble salts and complexes to which a precipitating reagent is added. Furthermore, it is possible to dope the oxidic catalyst precursor on the surface with metal salts prior to the reduction by bringing said metal salts into contact with the mixed oxide for a certain time, for example in aqueous solution. Also after reduction of the catalyst precursor and even during the hydrogenation reaction, the catalyst already prepared via the reduction of a catalyst precursor can still be doped in the same manner. The catalyst precursor and/or also the catalyst may already be doped with doping elements.

The catalyst precursor which is as a rule obtained in powder form can be subjected to shaping or absorbed on porous and surface-active materials (provision of support) prior to the reduction. Customary methods of shaping and providing a support are described, for example, in Ullmann [Ullmann's Encyclopedia Electronic Release 2000, Chapter: “Catalysis and Catalysts”, pages 28-32]. It is also possible for suitable substances to be applied to a support and reacted there, the catalyst precursor forming.

The reduction of the catalyst precursor can be effected in the liquid in which the catalyst precursor is suspended. The reduction in the liquid can be effected, for example, in a stirred autoclave, a packed bubble column, a circulation reactor or a fixed-bed reactor.

The reduction can also be carried out in the dry state as powder in an agitated or unagitated reducing oven or in a fixed bed or in a fluidized bed. In a preferred embodiment, the reduction of the catalyst precursor is carried out in a liquid in which the catalyst precursor is suspended.

Suitable liquids for suspending the catalyst precursor are water or organic solvents, e.g. ethers, such as methyl tert-butyl ether, ethyl tert-butyl ether or tetrahydrofuran (THF), alcohols, such as methanol, ethanol or isopropanol, hydrocarbons, such as hexane, heptane or raffinate cuts, aromatics, such as toluene, or amides such as dimethylformamide or dimethylacetamide, or lactams, such as N-methylpyrrolidone, N-ethylpryrrolidone, N-methylcaprolactam or N-ethylcaprolactam. Other suitable liquids are suitable mixtures of the abovementioned solvents.

Preferred liquids comprise products from the hydrogenation to be carried out. Liquids which are the product of the hydrogenation to be carried out are particularly preferred.

In a further preferred variant, the catalyst precursor is suspended in a liquid which comprises no water.

In the reduction of the catalyst precursor in suspension, the temperatures are in general in a range of from 50 to 300° C., in particular from 100 to 250° C., particularly preferably from 120 to 200° C.

The reduction in suspension is carried out as a rule at a pressure of from 1 to 300 bar, preferably from 10 to 250 bar, particularly preferably from 30 to 200 bar, the pressure data here and below being based on the measured absolute pressure.

A suitable reducing agent is hydrogen or a gas comprising hydrogen or a hydride ion source.

In general, technically pure hydrogen is used. The hydrogen may also be used in the form of a gas comprising a hydrogen, i.e. in mixtures with other inert gases, such as nitrogen, helium, neon, argon or carbon dioxide. The hydrogen stream may also be passed back as recycled gas into the reduction, if appropriate mixed with fresh hydrogen and, if appropriate, after removal of water by condensation.

The reduction of the dry, generally pulverulent catalyst precursor can be carried out at elevated temperature in an agitated or unagitated reduction oven. Reduction of the catalyst precursor is effected as a rule at reduction temperatures of from 50 to 600° C., in particular from 100 to 500° C., particularly preferably from 150 to 400° C.

The operating pressure is as a rule from 1 to 300 bar, in particular from 1 to 200 bar, particularly preferably from 1 to 10 bar, it being possible for a hydrogen stream or a stream which comprises hydrogen and, as described above, may also comprise added amounts of other inert gasses to be passed through or over the catalyst bed. In this embodiment, too, the hydrogen stream can be passed back as recycled gas into the reduction, if appropriate mixed with fresh hydrogen and, if appropriate, after removal of water by condensation.

The reduction is preferably carried out in such a way that the degree of reduction is at least 50%. A comparison of the decrease in mass of dry catalyst precursor with dry, reduced catalyst is carried out as a method of measurement for the degree of reduction, in which comparison these samples are reduced from room temperature to 900° C. in a gas stream comprising hydrogen, and the integral of the decrease in mass is recorded. The degree of reduction is calculated from the ratio of the decreases in weight as follows: degree of reduction [%]=100·(1−(decrease in weight_{reduced catalyst}/decrease in weight_{oxidic precursor}))

During the reduction, a solvent may be added in order to remove resulting water of reaction. Here, the solvent can also be fed in supercritically.

Suitable solvents may be the same as those which, as described above, are suitable for suspending the catalyst. Preferred solvents are ethers, such as methyl tert-butyl ether, ethyl tert-butyl ether or tetrahydrofuran, alcohols such as methanol, ethanol or isopropanol, hydrocarbons, such as, hexane, heptane or raffinate cuts, aromatics, such as toluene, or amides, such as dimethylformamide or dimethylacetamide, or lactams, such as N-methylpyrrolidone, N-ethylpyrrolidone, N-methylcaprolactam or N-ethylcaprolactam. Methanol or tetrahydrofuran is particularly preferred. Suitable mixtures are also suitable solvents. The abovementioned reaction conditions for the reduction of the catalyst precursor are generally applicable, for example for stirred autoclaves, fluidized beds or fixed-bed processes. The catalyst according to the invention can also be prepared by reduction with a hydride ion source in a solvent, starting from the catalyst precursor. Suitable hydride ion sources are complex hydrides, such as LiAlH₄ or NaBH₄. Suitable solvents are ethers, such as methyl tert-butyl ether, ethyl tert-butyl ether or tetrahydrofuran. Hydrocarbons, such as hexane, heptane or raffinate cuts, or aromatics, such as toluene. Tetrahydrofuran is particularly preferred. Suitable mixtures are also suitable solvents.

With the use of a hydride ion source, the reduction is preferably carried out at temperatures of 10-200° C. at the corresponding autogenous pressure of the system.

The reduction of the catalyst precursor can preferably be carried out up to a degree of reduction of from 50 to 100%.

After the reduction, the catalyst can be handled and stored under an inert gas, such as nitrogen, or under an inert liquid, for example in alcohol, water or the product of the respective reaction for which the catalyst is used. However, after the reduction, the catalyst can also be passivated, i.e. provided with a protective oxide layer, using a gas stream comprising oxygen, such as air or a mixture of air with nitrogen.

Below, the term catalyst designates a catalyst which was prepared according to the invention by reducing the catalyst precursor described, or a catalyst which, as described above, was passivated with a gas stream comprising oxygen after the activation.

The storage of the catalyst under inert substances or the passivation of the catalyst permits uncomplicated and safe handling and storage of the catalyst. If appropriate, before the beginning of the actual reaction, the catalyst must then be freed from the inert liquid or the passivating layer must be eliminated, for example by treatment with hydrogen or with gas comprising hydrogen.

The catalysts according to the invention can be used in a process for the hydrogenation of compounds which comprise at least one unsaturated carbon-carbon, carbon-nitrogen or carbon-oxygen bond, or for the partial or complete nuclear hydrogenation of compounds comprising aromatics.

Suitable compounds are as a rule compounds which comprise at least one or more carboxamido groups, nitrile groups, imino groups, enamine groups, azine groups or oxime groups, which are hydrogenated to amines.

Furthermore, in the process according to the invention, compounds which comprise at least one or more carboxylic ester groups, carboxyl groups, aldehyde groups or keto groups which are hydrogenated to alcohols can.

Other suitable compounds are aromatics, which can be converted into unsaturated or saturated carbocycles or heterocycles.

Particularly suitable compounds which can be used in the process according to the invention are organic nitrile compounds. These can be hydrogenated to primary amines.

Suitable nitrites are acetonitrile for the preparation of ethylamine, propionitrile for the preparation of propylamine, butyronitrile for the preparation of butylamine, lauronitrile for the preparation of laurylamine, stearylnitrile for the preparation of stearylamine, N,N-dimethylaminopropionitrile (DMAPN) for the preparation of N,N-dimethylaminopropylamine (DMAPA) and benzonitrile for the preparation of benzylamine. Suitable dinitriles are adipodinitrile (ADN) for the preparation of hexamethylenediamine (HMD) and/or aminocapronitrile (ACN), 2-methylglutarodinitrile for the preparation of 2-methyl-glutarodiamine, succinonitrile for the preparation of 1,4-butanediamine and suberodinitrile for the preparation of octamethylenediamine. Cyclic nitriles, such as isophoronenitrilimine(isophoronenitrile) for the preparation of isophoronediamine and isophthalodinitrile for the preparation of meta-xylylenediamine, are furthermore suitable. Also suitable are α-aminonitriles and β-aminonitriles, such as aminopropionitrile for the preparation of 1,3-diaminopropane, or ω-aminonitriles, such as aminocapronitrile for the preparation of hexamethylenediamine. Further suitable compounds are so-called “Strecker nitriles”, such as iminodiacetonitrile for the preparation of diethylenetriamine. Dinitrotoluene for the preparation of toluidinediamine is also suitable. Further suitable nitriles are β-aminonitrinles, for example adducts of alkylamines, alkyldiamines or alkanolamines and acrylonitrile. Thus, adducts of ethylenediamine and acrylonitrile can be converted into the corresponding diamines. For example, 3-[(2-aminoethyl)amino]propionitrile can be converted into 3-(2-aminoethyl)amino-propylamine and 3,3′-(ethylenediimino)bispropionitrile or 3-[2-(3-aminopropylamino)ethylamino]propionitrile can be converted into N,N′-bis(3-aminopropyl)ethylenediamine.

N,N-Dimethylaminopropionitrile (DMAPN) for the preparation of N,N-dimethylaminopropylamine (DMAPA) and adipodinitrile (AND) for the preparation of hexamethylenediamine (HMD) are particularly preferably used in the process according to the invention.

Hydrogen, a gas comprising hydrogen or a hydride ion source can be used as a reducing agent.

The hydrogen used for the hydrogenation is used in general in a relatively large stoichiometric excess of from 1 to 25 times, preferably from 2 to 10 times, or in stoichiometric amounts. It may be passed back as recycled gas into the reaction. The hydrogen is used in general in technically pure form. The hydrogen may also be used in the form of a gas comprising hydrogen, i.e. in admixtures with other inert gases, such as nitrogen, helium, neon, argon or carbon dioxide.

The hydrogenation can also be effected using a hydride ion source. Suitable hydride ion sources are complex hydrides, such as LiAlH₄ or NaBH₄.

In a process for preparation of amines by reduction of nitriles, the hydrogenation can be effected with the addition of ammonia. Ammonia is used as a rule in molar ratios of from 0.5:1 to 100:1, preferably from 2:1 to 20:1, relative to the nitrile group. The preferred embodiment is a process in which no ammonia is added.

The hydrogenation can be carried out in the presence of a liquid.

The liquid may be the same liquid in which, as described above, the catalyst precursor was reduced or suspended.

Suitable liquids are, for example, C₁- to C₄-alcohols, C₄- to C₁₂-dialkyl ethers or cyclic C₄- to C₁₂-ethers, such as tetrahydrofuran or tert-butyl methyl ether. Suitable liquids may also be mixtures of the abovementioned liquids. The liquid may also be the product of the hydrogenation.

In a preferred embodiment, the hydrogenation is carried out in an anhydrous liquid.

The catalyst can be freed from the inert liquid or passivating layer before the beginning of the hydrogenation. This is effected, for example, by treatment with hydrogen or a gas comprising hydrogen. Preferably, the hydrogenation is carried out directly after the reduction of the catalyst precursor in the same reactor as that in which the reduction was also effected.

The hydrogenation is carried out as a rule at a pressure of from 1 to 300 bar, in particular from 5 to 200 bar, preferably from 8 to 85 bar and particularly preferably from 10 to 65 bar. Preferably, the hydrogenation is carried out at a pressure of less than 65 bar as a low-pressure process.

The temperature is as a rule in a range of from 40 to 250° C., in particular from 60 to 160° C., preferably from 70 to 150° C., particularly preferably from 80 to 130° C.

The hydrogenation can be effected, for example, in the liquid phase in a stirred autoclave, a bubble column, a circulation reactor, such as, for example, a jet loop, or a fixed-bed reactor.

The catalyst can be separated from the product by methods known to the person skilled in the art, for example filtration or a settling method.

The hydrogenation can also be carried out in the gas phase in a fixed-bed reactor or fluidized-bed reactor. Customary reactors for carrying out hydrogenation reactions are described, for example, in Ullmann's Encyclopedia [Ullmann's Encyclopedia Electronic Release 2000, chapter: Hydrogenation and Dehydrogenation, pages 2-3].

The hydrogenation is preferably carried out in suspension.

In a particular embodiment, mostly for reasons of process simplification, the hydrogenation is carried out in the same reaction vessel in which the reduction of the catalyst precursor is also effected.

The hydrogenation processes can be carried out batchwise, semi-continuously or continuously. The hydrogenation processes are preferably carried out semi-continuously or continuously.

The activity and/or selectivity of the catalysts according to the invention may decrease with the increasing on-stream time. Accordingly, a process for regenerating the catalysts according to the invention was found, in which the catalyst is treated with a liquid. The treatment of the catalyst with a liquid should result in the removal of any adhering compounds which block active sites of the catalyst. The treatment of the catalyst with a liquid can be effected by stirring the catalyst in a liquid or by washing the catalyst in the liquid, it being possible, after the treatment is complete, for the liquid to be separated from the catalyst by filtration or decanting together with the impurities removed.

Suitable liquids are as a rule the product of the hydrogenation, water or an organic solvent, preferably ethers, alcohols or amides.

In a further embodiment, the treatment of the catalyst with liquid can be effected in the presence of hydrogen or of a gas comprising hydrogen.

This regeneration can be carried out at elevated temperature, as a rule from 20 to 250° C. It is also possible to dry the spent catalyst and to oxidize adhering organic compounds under air to give volatile compounds, such as CO₂. Before further use of the catalyst in the hydrogenation, said catalyst must as a rule be activated as described above after oxidation is complete.

In the regeneration, the catalyst can be subsequently doped with a compound of the element b). The subsequent doping can be effected by impregnating or wetting the catalyst with a water-soluble base of the element b).

An advantage of the invention is that, by using the catalyst according to the invention, the requirement in terms of apparatus and capital costs and the operating costs for plants in the case of hydrogenation processes are reduced. In particular, the capital costs increase with increasing operating pressure and with the use of solvents and additives. Since the hydrogenation process according to the invention can also be operated in the absence of water and ammonia, process steps for separating the water and ammonia from the reaction product (distillation) are dispensed with or simplified. Furthermore, because of the absence of water and ammonia, the existing reactor volume can be better utilized since the volume which becomes free can be used as additional reaction volume.

Because the reduction of the catalyst precursor according to the invention can be carried out in a liquid, catalyst particles having a smaller size and larger surface area can be obtained.

The invention is explained in the following examples.

### DEFINITION

The catalyst space velocity is stated as the quotient of amount of product and the product of catalyst mass and time.

Catalyst space velocity=amount of product/(catalyst mass·reaction time)

The unit of the catalyst space velocity is stated in [kg_{product}/(kg_{cat}·h)] or [g_{product}/(g_{cat}·h)].

The stated selectivities were determined by gas chromatographic analyses and calculated from the area percentages.

The conversion of starting material C(S) is calculated according to the following formula:

$$C ( S ) = \frac{A \% {( S )}_{Start} - A \% {( S )}_{End}}{A \% {( S )}_{Start}}$$

The yield of product Y(P) is obtained from the area percentages of the product signal.

Y(P)=A%(P),

the area percentages A %(I) of a starting material (A %(S)), product (A %(P)), a byproduct (A %(B)) or very generally a substance i (A %(i)) being obtained from the quotient of the area A(i) below the signal of the substance i and the total area Aₜₒₜₐₗ, i.e. sum of the areas below the signals i, multiplied by 100:

$$A \% ( i ) = \frac{A ( i )}{{A}_{total}} \cdot 100 = \frac{A ( i )}{{\sum }_{i} A ( i )} \cdot 100$$

The selectivity of the starting material S(S) is calculated as the quotient of product yield Y(P) and conversion of starting material C(S):

$$S ( S ) = \frac{Y ( P )}{C ( S )}$$

If dimethylamine (DMA) was added to DMAPN, the stated area percentages are based on the total area without the area below the DMA signal.

$${A}_{total} = {\sum }_{i} A ( i ) where i \neq D M A$$

This is effected on the assumption that the DMA found in the product has not been formed by cleavage of the starting material but originates exclusively through the prior addition.

### Abbreviations Used:

g: gram
% by weight: percent by weight
h: hour(s)
kg: kilogram
min.: minute
ml: milliliter
ppm: parts per million
% by volume: percent by volume
XRD: X-ray diffraction
ADN: adipodinitrile
ACN: aminocapronitrile
DMA: dimethylamine
DMAPA: N,N-dimethylaminopropylamine
DMAPN: dimethylaminopropionitrile
HMD: hexamethylenediamine
THF: tetrahydrofuran

### EXAMPLE 1

### A) Preparation of a Catalyst According to the Invention

80 g of THF and 3.0 g of LiCoO₂ were combined in a high-pressure autoclave. The autoclave was closed, the mixture was blanketed and hydrogen was forced in to 10 bar. Heating to 150° C. was effected under autogenous pressure and with stirring. On reaching this temperature, hydrogen was forced in to 100 bar. Reduction was then effected for 12 h. Thereafter, the autoclave was allowed to cool and was let down to about 36 bar.

### B) Hydrogenation of DMAPN

Directly after the catalyst preparation (1A) 0.44 ml/min of crude DMAPN which comprised 2.5% by weight of DMA was pumped into the reactor at a temperature of 100° C. and a pressure of 36 bar, and the pressure was kept approximately constant by forcing in further hydrogen. This corresponds to a catalyst space velocity of 7.5 g of DMAPN/(g of LiCoO₂·h). In the period of from 8 to 20 h the selectivity of the DMAPA obtained in the crude discharge was from 98.7 to 99.6%. After 20 h the space velocity was doubled. This resulted in a decline in the conversion to 95% and a lowering of the selectivity to 96.1%. On increasing the temperature to 140° C. and the pressure to 60 bar, it was possible to increase the conversion again to full conversion, the selectivity increasing to 98.8% (53 h). An analysis of the discharge (from 62 to 74 h) for Li and Co was negative, and less than 1 ppm of Li and Co were detected.

### EXAMPLE 2

### A) Preparation of a Catalyst According to the Invention

1.5 g of LiCoO₂ were combined with 35 g of THF in a stirred autoclave and activated at 150° C. and with 100 bar hydrogen for 24 h with vigorous stirring. After the stirring, the autoclave was allowed to cool and was let down to 10 bar.

### B) Hydrogenation of DMAPN

Directly after the catalyst preparation (2A), a temperature of 1000° C. was established. After this temperature had been reached, a pressure of 36 bar was established by forcing in hydrogen. Thereafter, 24 g of pure DMAPN (catalyst space velocity=7 g of DMAPN/(g LiCoO₂·h)) were metered over 2 h with stirring, and the pressure was kept approximately constant by forcing in further hydrogen. After 2 h, the metering was switched off, a waiting time of one minute was allowed and then 17 g of the reactor contents were removed. This procedure was repeated twice more, 22 g of the reactor content being removed the second time and 27 g being removed the third time. In each case, the analyses showed full conversion and a selectivity of 99.7%, based on DMAPA. An analysis of the last discharge for Li and Co gave <1 ppm of Co and about 1 ppm of Li.

Examples 1 and 2 demonstrate the high efficiency of the catalysts according to the invention which were prepared from the catalyst precursor LiCoO₂ over a relatively long period. Furthermore, it was possible to show that the Li present in the precursor stage was not converted into a soluble form by the reduction and it was discharged in a continuous process. A further advantage evident from the examples is the fact that the catalyst can be activated in standard apparatuses under mild conditions. The water present at the beginning of the experiment is not required for the activity of the catalyst according to the invention since it is continuously removed and the catalyst nevertheless remains active.

### EXAMPLE 3

### A) Preparation of a Catalyst According to the Invention

100 g of THF and 12 g of LiCoO₂ were combined in a high-pressure autoclave. The autoclave was closed, the mixture was blanketed and hydrogen was forced in to 10 bar. Heating to 200° C. was effected under autogenous pressure and with stirring. On reaching this temperature, hydrogen was forced in to 100 bar. Reduction was then effected for 24 h. Thereafter, the autoclave was allowed to coot and was let down under nitrogen. Thereafter the catalyst (3A) was filtered off in an apparatus under nitrogen excess pressure and washed with THF. The black paste thus obtained (33.8 g) had a dry mass fraction of about 37%.

### B) Hydrogenation of Unsaturated Substrates

The experiments 3.1 to 3.5 shown in table 1 were then carried out with the catalyst (3A).

TABLE 1
Hydrogenation of unsaturated substrates

| | | | | Amount of | | | | Initial | Amount |
|---|---|---|---|---|---|---|---|---|---|
| N^{o} | Procedure | Substrates | Catalyst | catalyst | Pressure | Temp. | S.V.¹ | amount | metered in |
| 3.1 | batch | acetonitrile | catalyst | 0.35 g | 30 bar | 100° C. | | 70 g | — |
| | | | 3A) | | | | | aceto- | |
| | | | | | | | | nitrile | |
| 3.2 | batch | cyclo- | catalyst | 0.7 g | 36 bar | 100° C. | | 70 g | — |
| | | hexanone | 3A) | | | | | cyclo- | |
| | | | | | | | | hexanone | |
| 3.3 | batch | cyclooctadiene | catalyst | 0.7 g | 36 bar | 140° C. | | 70 g | — |
| | | | 3A) | | | | | cyclo- | |
| | | | | | | | | octadiene | |
| 3.4 | fed-batch | ADN | catalyst | 2.4 g | 36 bar | 100° C. | 1.7 | 40 g THF | 24 g ADN |
| | | | 3A) | | | | | | in 6 h |
| 3.5 | fed-batch | DMAPN | catalyst | 2.23 g | 36 bar | 100° C. | 7 | 35 g | 48 g |
| | | | 3A) | | | | | DMAPA | DMAPN in |
| | | | | | | | | | 8 h |

¹Catalyst space velocity in kg of substance/[kg_{cat} * h]

After the preparation of the catalyst (3A), the amount of catalyst stated in the table was added to a stirred autoclave and the amount of initially taken substance stated in the table. The reactor was then adjusted to the temperatures stated in the table. On reaching this temperature, the pressure stated in the table was established by forcing in hydrogen.

In the “batch experiments” (3.1 to 3.3) hydrogenation was then effected after switching on the stirrer, and the pressure was kept approximately constant by forcing in further hydrogen. The duration of the hydrogenation is stated in the column “metering time/hydrogenation time” in table 2. In table 2 the conversions and selectivities of the products obtained are listed.

TABLE 2
Hydrogenation results

| | Metering time/ | | | |
|---|---|---|---|---|
| | hydrogenation | | | Product |
| N^{o} | time | Conversion | Product | selectivity |
| 3.1 | 10 h | 18.5% | ethylamine | 80.2% |
| 3.2 | 8 h | 99.96% | cyclohexanol | 98.3% |
| 3.3 | 10 h | 100% | cyclooctane | 62.4%¹ |
| 3.4 | 6 h | 99.5% | hexamethylenediamine | 98.3% |
| 3.5 | 8 h | 99.6% | DMAPA | 99.6% |

¹in addition to the product, mainly 36% of cyclooctaene was found

In the “fed batch experiments” (3.4 to 3.6), after the activation of the catalyst precursor with stirring the amount of said starting materials stated in the column “metered amount” was metered in with stirring, and the pressure was kept approximately constant by forcing in further hydrogen. The analytical results after the stated time are likewise stated in table 2.

Example 3 shows that very different compounds comprising unsaturated carbon-carbon, carbon-nitrogen or carbon-oxygen bonds can be hydrogenated with very good selectivities.

### EXAMPLE 4

### A) Preparation of a Catalyst According to the Invention

1) Doping of LiCoO₂ with Nickel

12 g of LiCoO₂ and 1.2 g of Ni(II) acetate tetrahydrate were vigorously stirred in 50 ml of demineralized water in a closed glass bottle for 10 h. Thereafter, the black powder (4A-1) was filtered off and washed with water and with THF.

### 2) Preparation of the Catalyst:

13.2 g of the catalyst precursor thus treated (from example 4A-1) were then reduced in 100 g of THF at 200° C. and 100 bar over 24 h in a 300 ml hydrogenation autoclave. After the reduction, 17.8 g of reduced, THF-moist catalyst were obtained by filtration. The catalyst thus obtained (4A-2) had a dry mass fraction of about 57%.

### B) Hydrogenation of DMAPN

2.2 g of the catalyst (4A-2) were then introduced into a stirred autoclave and a temperature of 100° C. was established. After reaching this temperature, a pressure of 36 bar was established by forcing in hydrogen. Thereafter, 48 g of pure DMAPN (catalyst space velocity=4.1 g DMAPN/(g_{cat}·h)) were metered with stirring over 8 h, and the pressure was kept approximately constant by forcing in further hydrogen. The sample after 8 h metering and hydrogenation gave 99.0% conversion and 99.7% selectivity, based on DMAPA.

Example 4 shows that the Ni-doped catalyst has a lower activity but a higher selectivity in the hydrogenation of DMAPN than the undoped catalyst from example 1A).

### EXAMPLE 5

### A) Use of a Catalyst According to the Invention for the Hydrogenation of ADN

6 g of LiCoO₂ were reduced as described in example 2A in 80 g of THF. 60 g of ADN were then metered in at 36 bar and 100° C. over 6 h. The hydrogen pressure was kept constant by continuously forcing in further hydrogen. After 6 h the ADN metering was stopped and hydrogenation was continued for a further 6 h. The gas chromatographic analysis of the sample after 6 h showed 99.8% conversion and 97.6% selectivity, based on HMD and ACN. 97.0% of HMD and 0.5% of ACN had been formed.

### COMPARATIVE EXAMPLE 1

### A) Preparation of a Comparative Catalyst

6 g of CO₃O₄ were combined with 80 g of THF in a high-pressure autoclave and activated at 200° C. and 100 bar H₂ for 12 h with vigorous stirring. After the stirring, the autoclave was allowed to cool to 100° C. and was let down to 36 bar.

### B) Hydrogenation of ADN

Directly after the preparation of the comparative catalyst (C1-A) 60 g of pure ADN (catalyst space velocity: 1.7 g of ADN/(g_{cat}·h)) were metered at 100° C. and 36 bar with stirring over 6 h, and the pressure was kept at 36 bar by forcing in further hydrogen. After 6 h, the metering was stopped and stirring was continued for a further 6 h under the same conditions. The gas chromatographic analysis of the sample after 6 h showed 57% conversion and 87.7% selectivity, based on HMD and ACN. 30.5% of HMD and 19.4% of ACN had been formed. The gas chromatographic analysis of the sample after 12 h showed 81.0% conversion and 88.5% selectivity, based on HMD and ACN. 44.4% of HMD and 27.2% of ACN had been formed.

Example 5 and comparative example 1 show that the catalyst which is prepared by reducing a catalyst precursor which comprises the mixed oxide structure according to the invention has advantages over a catalyst which was prepared by reducing a catalyst precursor which consists of pure cobalt oxide. At the same catalyst space velocity, the productivity of the catalyst according to the invention was much higher than that of the catalyst which was prepared from the pure cobalt oxide catalyst precursor. Even after a subsequent hydrogenation time of 6 h, this catalyst still did not achieve the conversion which had been achieved in the case of LiCoO₂ after only 6 h, although the reduction temperature had been about 50° C. higher.

### EXAMPLE 6

### A) Preparation of a Catalyst Precursor

Pulverulent magnesium carbonate and cobalt(II) carbonate hydrate (CAS 513-79-1) were thoroughly mixed in the ratio 0.5:1 [mol of Mg:mol of Co] and calcined in air in an oven. For this purpose, heating was effected for 2 h to 400° C. and this temperature was maintained for 2 h. In XRD (X-ray diffraction), the oxidic catalyst precursor thus obtained shows diffraction signals of CoO/MgO solid solutions and a spinel structure.

### B) Preparation of a Catalyst According to the Invention

In a heated reduction oven blanketed with nitrogen, the powder obtained from the calcination (example 6A) was gassed with a gas stream comprising 90% by volume of N₂ and 10% by volume of H₂ and heated to 300° C. in the course of 2 h, reduced for 16 h at this temperature and then cooled. After cooling, the hydrogen-containing atmosphere was exchanged for nitrogen. According to X-ray diffraction (XRD), the reduced catalyst thus obtained predominantly comprises cubic and hexagonal cobalt and CoO/MgO.

The reduced catalyst thus obtained (6B) was used as described below under 6C).

### C) Hydrogenation of DMAPN

3 g of the catalyst (6B) were combined with 35 g of DMAPA in a stirred autoclave. Hydrogen was forced in to 10 bar and heating to 100° C. was effected with gentle stirring. After reaching this temperature, further H₂ was forced in to 36 bar and the metering of 6 g/h of DMAPN was started. The hydrogen pressure was kept approximately constant by continuously forcing in further hydrogen. After 8 h, the metering was terminated and hydrogenation was continued for a further 3 h. A sample after 8 h showed 99.8% conversion and 99.3% selectivity. After 11 h, the conversion was 99.95% and the selectivity 99.2%.

### EXAMPLE 7

### A) Preparation of a Catalyst Precursor

Pulverulent lithium carbonate (CAS 554-13-2) and cobalt(II) carbonate hydrate (CAS 513-79-1) were thoroughly mixed in the ratio of 1:1 [mol of Li:mol of Co] and calcined in air in an oven. For this purpose, heating to 400° C. was effected in 2 h and this temperature was maintained for 2 h. The catalyst precursor thus obtained had an Li:Co ratio of 1:1 [mol:mol] (from elemental analysis) and a surface area of 34 m²/g (BET measurement). From the diffraction lines in the X-ray powder diffraction pattern (XRD, Cu—K-alpha radiation), it was concluded that the crystalline main constituent of this catalyst precursor is an LiCoO₂ mixed oxide.

### B) Preparation of a Catalyst According to the Invention

In a heated reduction oven blanketed with nitrogen, the powder obtained from the calcination (example 7A) was gassed with a gas stream comprising 90% by volume of N₂ and 10% by volume of H₂ and heated to 300° C. in the course of 2 h, reduced for 16 h at this temperature and then cooled. After cooling, the hydrogen-containing atmosphere was exchanged for nitrogen.

The reduced catalyst thus obtained (7B) was used as described under 7C).

For passivation of the catalyst, air was slowly added to the nitrogen atmosphere until the nitrogen had been completely exchanged for air.

The passivated catalyst thus obtained was used as described under 7D) and 7E).

### C) Semi-Batch Hydrogenation of DMAPN

A semi-batch experiment for DMAPN hydrogenation was carried out with 3.0 g of the catalyst from example 7B). 35 g of DMAPA were initially taken in a stirred autoclave and a temperature of 100° C. was established. After reaching this temperature, a pressure of 36 bar was established by forcing in hydrogen. Thereafter, 35 g of DMAPN (catalyst space velocity about 2 g of DMAPN/(g_{cat}·h)) were metered in with stirring over 8 h and the pressure was kept approximately constant by forcing in further hydrogen. The sample after 8 h metering and hydrogenation gave 99.9% conversion and 99.6% selectivity, based on DMAPA.

### D) Semi-Batch Hydrogenation of DMAPN

A semi-batch experiment for DMAPN hydrogenation was carried out with 3.0 g of the passivated catalyst from example 7B), 35 g of DMAPA were initially taken in a stirred autoclave and a temperature of 100° C. was established. After reaching this temperature, a pressure of 36 bar was established by forcing in hydrogen. Thereafter, 35 g of DMAPN (catalyst space velocity about 2 g of DMAPN/(g_{cat}·h)) were metered in with stirring over 8 h and the pressure was kept approximately constant by forcing in further hydrogen. The sample after 8 h metering and hydrogenation gave 99.9% conversion and 99.7% selectivity, based on DMAPA.

### E) Continuous Hydrogenation of DMAPN

The passivated catalyst from example 7B) was used in the continuous hydrogenation of DMAPN in suspension without preactivation. At a hydrogen pressure of 40 bar and 120° C., 2.5% by weight of catalyst and a space velocity of 1.2 kg of DMAPN/(kg_{cat}·h), the experiment was completed without signs of deactivation after 400 h at constant high DMAPN conversion of >99.9% with constant high selectivity of 99.5%.

Example 7 shows that the catalyst can be used in completely reduced or passivated form, separate activation of the passivated catalyst before the beginning of the hydrogenation not being absolutely essential.

Example 7 also shows that the catalyst is also suitable for use in continuous processes.

### EXAMPLE 8

### A) Preparation of a Catalyst Precursor

Pulverulent lithium carbonate (CAS 554-13-2) and cobalt(II) carbonate hydrate (CAS 513-79-1) were thoroughly mixed in the ratio 0.8:1 [mol of Li:mol of Co] and calcined in air in an oven. For this purpose, heating to 400° C. was effected in the course of 2 h and this temperature was maintained for 2 h. From the diffraction lines of the catalyst precursor thus obtained (8A) in the X-ray powder diffraction pattern (XRD, Cu—K-alpha radiation) it was possible to conclude that, in addition to the crystalline main constituent, a non-stoichiometric LiₓCo_{(1+x/3)}O₂ mixed oxide, a little CO₃O₄ is also present.

### B) Preparation of a Catalyst According to the Invention

In a heated reduction oven blanketed with nitrogen, the catalyst precursor obtained from the calcination (8A) was gassed with a gas stream comprising 90% by volume of N₂ and 10% by volume of H₂ and heated to 300° C. in the course of 2 h, reduced for 16 h at this temperature and then cooled. After cooling, the hydrogen-containing atmosphere was exchanged for nitrogen.

The reduced catalyst thus obtained (8B) was used as described under C).

### C) Hydrogenation of DMAPN

A semi-batch experiment for DMAPN hydrogenation was carried out with 3.0 g of the catalyst (8B). 35 g of DMAPA were initially taken in a stirred autoclave and a temperature of 100° C. was established. After reaching this temperature, a pressure of 36 bar was established by forcing in hydrogen. Thereafter, 35 g of DMAPN (catalyst space velocity about 2 g of DMAPN/(g_{cat}·h)) were metered in with stirring over 8 h and the pressure was kept approximately constant by forcing in further hydrogen. The sample after 8 h metering and hydrogenation gave 99.8% conversion and 99.8% selectivity, based on DMAPA.

Example 8 clearly shows that catalyst precursors which comprise a mixed oxide predominantly but not exclusively are also suitable according to the invention.

1-12. (canceled)

13. A catalyst obtained by reducing a catalyst precursor comprising a) cobalt and b) one or more elements of (1) the alkali metal group, (2) the alkaline earth metal group, (3) the group consisting of the rare earths, (4) zinc, or (5) mixtures thereof wherein a) and b) are present in said catalyst precursor at least partly in the form of their mixed oxides.

14. The catalyst of claim 13, wherein said catalyst precursor is LiCoO₂.

15. The catalyst of claim 14, wherein said LiCoO₂ is obtained from the recycling of batteries.

16. The catalyst of claim 13, wherein said reduction of said catalyst precursor is performed in a liquid.

17. A process for preparing a catalyst, wherein a catalyst precursor comprising a) cobalt and b) one or more elements of (1) the alkali metal group, (2) the alkaline earth metal group, (3) the group consisting of the rare earths, (4) zinc, or (5) mixtures thereof is reduced, wherein a) and b) are present in said catalyst precursor at least partly in the form of their mixed oxides.

18. The process of claim 17, wherein said catalyst precursor is LiCoO₂.

19. A process for hydrogenating a compound which comprises at least one unsaturated carbon-carbon, carbon-nitrogen, or carbon-oxygen bond, or partially or completely nuclear hydrogenating a compound which comprises an aromatic group, comprising hydrogenating said compound which comprises at least one unsaturated carbon-carbon, carbon-nitrogen, or carbon-oxygen bond, or partially or completely nuclear hydrogenating said compound which comprises an aromatic group in the presence of the catalyst of claim 13.

20. A process for preparing a primary amine from a compound comprising at least one nitrile group comprising hydrogenating said compound comprising at least one nitrile group in the presence of the catalyst of claim 13.

21. The process of claim 19, wherein said hydrogenation is carried out at low-pressure.

22. A process for regenerating the catalyst of claim 13, comprising treating said catalyst with a liquid.
